(19) United States
(12) Patent Application Publication
(10) Pub. No.: US 2008/0299048 A1
(43) Pub. Date: Dec. 4, 2008

(54) MIXED DRUG AEROSOL COMPOSITIONS

(75) Inventors: Ron L. Hale, Woodside, CA (US); Kathleen Simis, San Mateo, CA (US); Amy T. Lu, Los Altos, CA (US); Joshua D. Rabinowitz, Princeton, NJ (US); Krishnamohan Sharma, Milpitas, CA (US); William Shen, New York, NY (US); Justin Virgili, Berkeley, CA (US)

Correspondence Address: SWANSON & BRATSCHUN, L.L.C, 8210 SOUTHPARK TERRACE, LITTLETON, CO, 80120, US

(73) Assignee: ALEXZA PHARMACEUTICALS, INC., Palo Alto, CA (US)

(21) Appl. No.: 11/964,630

(22) Filed: Dec. 26, 2007

Related U.S. Application Data
(60) Provisional application No. 60/871,693, filed on Dec. 22, 2006.

(51) Int. Cl. A61K 9/12

(52) U.S. Cl. 424/45

(57) ABSTRACT

The present invention pertains to aerosols which comprise a first compound which is physiologically active and a second compound which is different from the first compound. Such aerosols may be produced “on demand” and can be used to control drug release, to improve vaporizability, or to reduce, modify or eliminate undesirable taste associated with a drug aerosol. The present invention also pertains to methods for producing such aerosols.

### CROSS-REFERENCE TO RELATED APPLICATIONS

This application claims priority to U.S. provisional application Ser. No. 60/871,693 entitled “Drug and Excipient Aerosol Composition to Modulate Pharmacokinetic Absorption, Improve Vaporizability, and/or Impart Taste Masking,” filed Dec. 22, 2006, Ron L. Hale, the entire disclosure of which is hereby incorporated by reference.

### FIELD OF THE INVENTION

The present invention pertains to aerosols which comprise a first compound which is physiologically active and a second compound which is different from the first compound. Such aerosols may be produced “on demand” and can be used to control drug release, to improve vaporizability, or to reduce, modify, or eliminate undesirable taste associated with a drug aerosol. The present invention also pertains to methods for producing such aerosols.

### BACKGROUND OF THE INVENTION

The “on-demand” generation of aerosols containing a first compound which is pharmaceutically active (i.e., a drug) and a second compound which is different from the first compound can be used to control pharmacokinetic profiles; to reduce, modify, or eliminate undesirable drug taste; deliver combination therapeutics; and/or to improve the vaporizability of an active pharmaceutical ingredient. Each of these applications may be expected to improve the therapeutic potential of the first compound by reducing side effects associated with high transient peak plasma concentrations, improving patient compliance by making the drug more palatable, improving the efficacy using another complementary drug, and/or allowing for the exploration of new delivery options for drugs that would otherwise not vaporize well.

Aerosols of the present invention comprise a first compound which is pharmaceutically active and a second compound which is different from the first compound. Typically, the second compound is inert. However, in some embodiments, the second compound also may be pharmaceutically active. The aerosol may comprise heterogeneous particles (i.e., particles that contain more than one type of compound, e.g., a first compound and a second compound), unary particles (i.e., particles that contain a single type of compound, e.g., either the first compound or the second compound), or a combination of heterogeneous particles and unary particles. In some embodiments, aerosols of the invention comprise two or more types of unary particles (e.g., unary particles containing a first compound and unary particles containing a second compound).

Heterogeneous aerosol particles may be particularly beneficial for controlling the pharmacokinetic profile of a drug. The drug from heterogeneous particles may be more slowly absorbed by the lung as compared to unary particles containing only the drug. Controlling the pharmacokinetic profile of a drug by suppressing pulmonary absorption would be beneficial for drugs that have side effects associated with transient spikes of the drug in systemic circulation or for locally acting drugs used in the treatment of lung-specific diseases.

It is believed that heterogeneous aerosol particles may reduce transient high levels of drug in the systemic blood circulation, which has been linked to cardiovascular risks. Other drugs of interest that would benefit from a reduction in side effects due to a sustained pulmonary absorption profile include citalopram (OCD/depression), triamterene (cystic fibrosis), and isoniazid (antituberculotic). For example, triptans may be a desirable drug class for administration via heterogeneous aerosol particles because such particles may exhibit a slight delay in the pharmacokinetic profile, which may alleviate concerns regarding pulmonary triptan delivery and cardiac safety.

Heterogeneous aerosol particles may also show greater efficacy than unary aerosol particles in the treatment of lung-specific diseases, such as cystic fibrosis (CF) or tuberculosis, because of their potentially sustained, lung-directed mechanism of action. In this case, controlled pharmacokinetic profiles will increase the residence time of locally acting drugs, ensuring that more drug is delivered to pulmonary tissue and less into the systemic circulation. By reducing the amount of drug in systemic circulation, patient safety may be improved. Other locally acting drugs that would benefit from increased pulmonary residence time via heterogeneous aerosol particles include anti-inflammatory steroids used in the treatment of asthma.

In the case of potentially addictive drugs, another benefit of heterogeneous aerosol particles may be to reduce the drug's addictive potential by reducing transient high levels of drug delivered to the brain.

Another benefit of heterogeneous aerosol particles is the potential for reducing, modifying, or eliminating undesirable taste associated with the first compound, thereby improving patient compliance. Heterogeneous particles may have less undesirable tasting drug on the surface of the particle that would be exposed to taste receptors than particles that containing drug only. Certain second compounds may simply provide better taste, thereby masking the drug taste, or may reduce or eliminate drug taste by blocking taste receptors. In addition, certain second compounds may neutralize electrostatic properties that can contribute to undesirable taste reception.

Aerosols comprising unary aerosol particles allow for combination therapies (two or more complementary drugs), provide taste attenuation, and may also be beneficial for improving the vaporizability of certain thermally labile compounds. Aerosols comprising unary particles include aerosols comprising complementary active compounds. For example, the aerosol may contain unary particles containing an anti-inflammatory steroid and unary particles containing a bronchodilator. Aerosols comprising unary particles advantageously may be used to improve the vaporizability of a drug. For example, an aerosol may comprise unary particles containing a high vapor pressure compound, such as caffeine, and unary particles containing a sublimable compound, such as theobromine or menthol.

Several methodologies have been proposed to control the absorption kinetics of respirable aerosol particles. For example, phospholipids endogenous to the lung have been used to encapsulate both hydrophilic and lipophilic drugs (Schreier et al., J. Controlled Release, vol. 24, pp. 209-223 (1993); Kellaway et al., Adv. Drug Del., Rev. 5, pp. 149-161 (1990)). Large microporous particles (˜10 μm) have been produced for the purpose of sustained release (Edwards et al., J. Appl. Physiol., Vol. 85, pp. 379-385 (1997); Edwards et al., Science, Vol. 276, pp. 1868-1871 (1998)).

However, inherent to both the liposomal encapsulation and large microporous particle approaches are concerns regarding manufacturing complexity and shelf-life stability. From a manufacturing standpoint, both formulations require complex and time-consuming preparation. Liposomal encapsulation involves a freeze-thaw method (Lange et al., J. Pharm. Sci., Vol. 90, pp. 1647-1657 (2001)), extrusion (Fielding et al., Pharm. Res., Vol. 9, pp. 220-223 (1992)), or sonication, while large microporous particles are made from a double-emulsion solvent evaporation process.

Liposomal formulations are typically delivered from nebulizers, and the liposomal vesicles can be damaged by high shear forces and entrapped drug may leak back into the supernatant. In addition, vesicle disruption could occur during droplet impact on baffles, again causing drug leakage (Finlay, The mechanics of inhaled pharmaceutical aerosols: An introduction, Ch. 8, Academic Press, London (2001)). The stability of existing sustained release formulations is also problematic. Microporous particles are delivered as dry powders and particle agglomeration during storage and inspiration can reduce drug bioavailability.

Another formulation approach to control drug release rates in the lungs involves coating of drug aerosol particles with hydrophobic materials (Pillai et al., J. Aerosol Sci., Vol. 25, pp. 461-477 (1994); Pillai et al., J. Appl. Physiol., Vol. 84, pp. 717-725 (1998)). In this approach, the drug aerosol droplets were first generated from a jet nebulizer. They were then dried, concentrated, and subsequently condensation-coated with paraffin wax or lauric acid by passing drug aerosol particles through a chamber containing paraffin wax or lauric acid vapor. By changing the wax or lauric acid vapor pressure, the mass ratio between drug and wax or lauric acid (or the thickness of the coating) can be varied. Using a canine model, Pillai et al. were able to show that paraffin (Pillai, 1998) and lauric acid (Pillai, 1994) coated disodium fluoresceine particles induced a two to four fold increase in absorption half-time over that of uncoated particles. These studies suggest that excipients with lower polar surface areas and less hydrogen bonding potential (i.e., more hydrophobic) may slow the pulmonary absorption rate of rizatriptan. However, they also illustrate the complex manufacturing procedures required to generate the mixed composition aerosol particles. (See also U.S. Patent Publication No. 2004/0185170, of Chungi et al., for a similar method of coating drug particles (beads, granules, pellets, etc.) via vapor deposition.)

### SUMMARY OF THE INVENTION

The present invention overcomes the manufacturing complexity and shelf-life stability issues encountered with prior mixed particle aerosol compositions, making it possible to generate mixed particle aerosol compositions “on demand” using a simple, inexpensive manufacturing method.

The present invention pertains to aerosols which comprise a first compound which is physiologically active and a second compound that is different from the first compound. Such aerosols may be produced “on demand” and can be used to control drug release, to improve vaporizability, or to reduce, modify, or eliminate undesirable taste associated with a drug aerosol. The present invention also pertains to methods for producing such aerosols.

In some embodiments, the aerosols comprise unary particles containing only a first compound and unary particles containing only a second compound. In some embodiments, the mass median aerodynamic diameter (MMAD) of the two populations of unary particles may be different. For example, the MMAD of the unary particles containing only the first compound may be within the range of 0.1 μm to 20 μm; 0.5 μm to 10 μm; 1 μm to 5 μm; or 1 μm to 3 μm. The MMAD of the unary particles containing only the second compound may also be within the range of 0.1 μm to 20 μm; 0.5 μm to 10 μm; 1 μm to 5 μm; or 1 μm to 3 μm. Alternatively, the MMAD of unary particles containing only the second compound may be, for example and without limitation, greater than 10 μm; greater than 15 μm; greater than 20 μm; greater than 30 μm, and so forth.

Typically, the aerosols comprise a certain fraction of heterogeneous particles which contain both the first compound and the second compound. Typically, the second compound is a physiologically inert additive or excipient. However, the second compound may also have pharmaceutical activity.

The mixed composition aerosols of the present invention can be designed to provide combination therapies (more than one active drug compound) or to impart improved vaporizability to an active drug compound. In particular, aerosols that comprise heterogeneous particles can be designed to enable delayed and/or sustained release pharmacokinetic profiles or to impart taste attenuation. As used herein, the term “taste attenuation” refers to the reduction, modification, or elimination of the undesirable taste associated with certain drugs.

Aerosols of the present invention are preferably generated by concurrent vaporization of the first compound and the second compound to create a vapor, followed by condensation of the vapor to form the aerosol. The production and administration of this mixed aerosol composition is “on demand”. For example, the first compound and the second compound may be deposited on a heating substrate in various coating configurations. For example, the first compound and the second compound may be co-deposited onto the same or separate area of the heating substrate surface simultaneously. Alternatively, the first compound and the second compound may be sequentially deposited onto the same area or onto separate areas of the substrate surface. For example, one of the compounds may be deposited onto a first surface of the substrate, and the other compound onto a second surface of the substrate. Alternatively, both compounds may be deposited onto the same surface of the substrate, with each compound deposited onto a separate area.

An electrical, chemical, or electrochemical heating mechanism (discussed, for example, in commonly assigned, copending U.S. application Ser. Nos. 10/850,895; 10/851,429; 10/851,883; 10/851,432; 10/861,554; and 10/917,735, the disclosures of which are hereby incorporated by reference in their entireties) may be activated to concurrently heat and vaporize the first compound and the second compound. The vapor comprising the first compound and the second compound is condensed to form an aerosol comprising heterogeneous aerosol particles, unary aerosol particles, or a combination of heterogeneous and unary aerosol particles.

A number of factors can affect the relative fractions of heterogeneous (multi-compound) aerosol particles and/or unary (single compound) aerosol particles in the mixed aerosol composition, including the relative mole fraction of excipient to drug, airflow dynamics, and relative chemistries. For example, larger ratios of second compound:first compound in the vapor phase, greater electronic interactions (e.g., ionic, Van der Waals, acid-base) between the first compound and second compound, and greater differences in vapor pressures between the first compound and second compound typically result in higher fractions of heterogeneous particles relative to unary particles in the mixed aerosol composition.

Aerosols of the present invention may comprise at least 10%, at least 20%, at least 30%, at least 40%, at least 50%, at least 75%, at least 90%, or at least 95% heterogeneous aerosol particles (i.e., aerosol particles which include both a first compound and a second compound). Aerosols of the present invention typically contain at least 1% of the second compound. The terms “first compound”, “physiologically active compound”, and “drug” are used interchangeably herein. The terms “second compound”, “physiologically inert compound” “additive” and “excipient” are used interchangeably herein.

The aerosolized particles typically have a mass median aerodynamic diameter (MMAD) within the range of 0.1 μm to 20 μm; preferably, within the range of 0.5 μm to 10 μm; and, most preferably, within the range of 1 μm to 5 μm, or 1 μm to 3 μm. Typically, the geometric standard deviation around the mass median aerodynamic diameter of the aerosol particles is less than 4. Preferably, the geometric standard deviation is less than 3. More preferably, the geometric standard deviation is less than 2.5, 2, 1.5, or, most preferably, approaches 1.

Also disclosed herein is a method of producing an aerosol comprising particles in which a fraction of the particles are heterogeneous particles (i.e., particles containing a first compound which is physiologically active and a second compound that is different from the first compound). The method includes the steps of: a) providing a heating substrate; b) coating at least a portion of a surface of the heating substrate with a first compound which is physiologically active and a second compound that is different from the first compound; and c) heating the substrate to a temperature sufficient to vaporize the first compound and the second compound (typically, at least 200° C.; preferably, at least 300° C.; most preferably, within the range of about 300° C. to about 450° C.), thereby generating a vapor comprising the first compound and the second compound. The vapor is condensed to form an aerosol. The first compound and the second compound are typically coated onto the heating substrate surface simultaneously. Alternatively, the first and second compound may be sequentially coated onto the same or separate areas of the substrate surface.

Coating of the first compound and second compound onto the heating substrate surface may be performed using a conventional coating technique known in the art, such as spray coating, dip coating, and inkjet printing. In the experimental examples described below, the first compound and second compound were applied to the exterior substrate surface(s) by spray coating using conventional ultrasonic spray coating techniques.

As discussed above, the relative mole fraction of the compound can dictate the relative fraction of heterogeneous and unary aerosol particles in the resulting aerosol, allowing further tailoring of the pharmacokinetic profile, taste attenuation, or improvement in vaporizability of the mixed aerosol composition. Higher excipient:drug ratios typically result in higher fractions of heterogeneous aerosol particles.

In preferred embodiments of the aerosol for the purpose of tailoring the pharmacokinetic profile, the first compound and the second compound (e.g., the absorption modifier) are typically coated onto the heating substrate at a mole ratio within the range of about 1:10 to about 10:1; preferably, within the range of about 1:5 to about 5:1; most preferably, within the range of about 1:1 to about 3:1 (second compound:first compound).

In preferred embodiments of the aerosol for the purpose of taste attenuation, the second compound (e.g., taste modifier) may be present at concentrations of as low as 1:1000 (second compound:first compound).

In preferred embodiments of the aerosol for the purpose of improving the vaporizability of the pharmaceutically active compound, the second compound may be present at concentrations of as low as 1:1000 to about 1:1 (second compound:first compound).

In preferred embodiments of the aerosol for the purpose of combination therapy, the optimal relative mole fraction of the first, pharmaceutically active compound and the second, pharmaceutically active compound are typically compound-specific, but most frequently will be in the range of 1:1 to 1:100 first compound:second compound.

Also contemplated herein is the generation of aerosols containing three or more different compounds. The three or more different compounds can all comprise pharmaceutically active compounds, or can be a combination of a pharmaceutically active compound or compounds with one or more inert compounds (e.g., additives or excipients).

### BRIEF DESCRIPTION OF THE DRAWINGS

Further features and advantages of the present invention will become apparent from the following description of various embodiments of the invention, as illustrated in the accompanying drawings in which:

FIG. 1A is an SEM photomicrograph of unary (single-compound) aerosol particles of rizatriptan.

FIG. 1B is an SEM photomicrograph of unary (single-compound) aerosol particles of palmitic acid.

FIG. 1C is an SEM photomicrograph of aerosol particles generated from a 1:3 mole ratio film of rizatriptan and palmitic acid.

FIG. 2 shows a Raman chemical analysis that compares the reference spectra of unary (single-compound) aerosol particles of rizatriptan and palmitic acid with the spectrum obtained from heterogeneous particles of palmitic acid and rizatriptan

FIG. 3 is a bar graph showing the laser desorption/ionization (ATOFMS-LDI) fractions of heterogeneous aerosol particles in samples generated from a 1:1 mole ratio of palmitic acid:rizatriptan, 100 and 500 μg drug loading; and a 3:1 mole ratio of palmitic acid:rizatriptan, 100 and 500 μg drug loading.

FIG. 4A is a line graph showing a comparison of the mass-based size distributions of the aerosol particles acquired from ATOFMS (rizatriptan, mixed) and APS (total size distribution) for a 1:1 mole ratio of palmitic acid:rizatriptan, 100 μg drug loading.

FIG. 4B is a line graph showing a comparison of the mass-based size distributions of the aerosol particles acquired from ATOFMS (rizatriptan, mixed) and APS (total size distribution) for a 3:1 mole ratio of palmitic acid:rizatriptan, 100 μg drug loading.

FIG. 5A is an ATOFMS spectrum generated from a rizatriptan aerosol sample.

FIG. 5B is an ATOFMS spectrum generated from a palmitic acid aerosol sample.

FIG. 5C is an ATOFMS spectrum generated from a 3:1 palmitic acid rizatriptan aerosol sample.

FIG. 6A shows a drug and a second compound coated on the same surface of a heating substrate, with the drug at the trailing edge of airflow across the substrate.

FIG. 6B shows a drug and a second compound coated on the same surface of a heating substrate, with the drug at the leading edge of airflow across the substrate.

FIG. 6C shows a drug coated onto the bottom surface of the substrate and a second compound coated onto the top surface of the substrate.

FIG. 7 is a bar graph showing aerosol purity of prochlorperazine (PCZ) and acesulfame (ACE) when vaporized from a heating substrate in the three different coating configurations shown in FIGS. 6A-6C.

FIG. 8A is an SEM photomicrograph of unary (single-compound) aerosol particles of acesulfame.

FIG. 8B is an SEM photomicrograph of unary (single-compound) aerosol particles of prochlorperazine.

FIG. 8C is an SEM photomicrograph of aerosol particles generated from a 1:1 mole ratio film of prochlorperazine and acesulfame.

### DETAILED DESCRIPTION OF THE PREFERRED EMBODIMENTS

According to the method of the present invention, physiologically active compounds with real or potential physiological activity can be volatilized along with a second, different compound, without medicinally significant degradation of the physiologically active compound. The resulting vapors can be controlled to produce mixed composition aerosols with average particle sizes in the range useful for administration of the aerosolized physiologically active compound to a patient. Physiologically active compounds which have been volatilized using the device and method of the present invention typically have a purity level of at least 90%; preferably, at least 95%; more preferably, at least 96%; and most preferably, at least 97%.

In the preferred embodiments of the present invention, compounds are volatilized into vapors, avoiding medicinally significant degradation and thus maintaining acceptable compound purity, by the steps of: (1) heating a substrate surface onto which a first which has physiological activity and a second compound that is different from the first compound have been coated to an elevated temperature for a limited time; and (2) under the conditions of step (1), simultaneously passing a gas (typically ambient air) across the substrate surface.

In commonly assigned, issued U.S. Pat. No. 7,090,830 and copending U.S. application Ser. No. 11/504,419, the disclosures of which are hereby incorporated by reference in their entireties, we disclosed devices and methods for generating and delivering aerosolized drugs by heating the drug to vaporize at least a portion of the drug, followed by mixing the resulting vapor with a gas, in a ratio, to form a desired particle size when a stable concentration of particles in the gas is reached. Pure drug was coated onto the surface of a heating unit which was used to heat the drug to the temperature required for volatilization of the drug. Pure (at least 90%; or preferably, at least 95%) drug was then delivered to the patient by inhalation of the aerosolized particles. However, as discussed above in the “Background of the Invention”, delivery of certain drugs could benefit from “on demand” generation of condensation aerosol particles containing a mixture of drugs and/or additives for the purpose of controlling pharmacokinetic profiles, improving drug palatability, and/or delivering combination therapeutics.

The present invention overcomes the manufacturing complexity and shelf-life stability issues encountered with prior mixed particle aerosol compositions, making it possible to generate mixed particle aerosol compositions “on demand” using a simple, inexpensive manufacturing method.

The present invention is broadly applicable to a wide variety of drugs. Typically, the drug belongs to one of the following classes: antibiotics, anticonvulsants, antidepressants, antiemetics, antihistamines, antiparkisonian drugs, antipsychotics, anxiolytics, drugs for erectile dysfunction, drugs for migraine headaches, drugs for the treatment of alcoholism, drugs for the treatment of addiction, muscle relaxants, nonsteroidal anti-inflammatories, opioids, other analgesics, and stimulants. Typically, when the drug is an antibiotic, it is selected from one of the following compounds: cefmetazole; cefazolin; cephalexin; cefoxitin; cephacetrile; cephaloglycin; cephaloridine; cephalosporins, such as cephalosporin C; cephalotin; cephamycins, such as cephamycin A, cephamycin B, and cephamycin C; cepharin; cephradine; ampicillin; amoxicillin; hetacillin; carfecillin; carindacillin; carbenicillin; amylpenicillin; azidocillin; benzylpenicillin; clometocillin; cloxacillin; cyclacillin; methicillin; nafcillin; 2-pentenylpenicillin; penicillins, such as penicillin N, penicillin O, penicillin S, penicillin V; chlorobutin penicillin; dicloxacillin; diphenicillin; heptylpenicillin; and metampicillin.

Typically, when the drug is an anticonvulsant, it is selected from one of the following compounds: gabapentin, tiagabine, and vigabatrin.
Typically, when the drug is an antidepressant, it is selected from one of the following compounds: amitriptyline, amoxapine, benmoxine, butriptyline, clomipramine, desipramine, dosulepin, doxepin, imipramine, kitanserin, lofepramine, medifoxamine, mianserin, maprotoline, mirtazapine, nortriptyline, protriptyline, trimipramine, viloxazine, citalopram, cotinine, duloxetine, fluoxetine, fluvoxamine, milnacipran, nisoxetine, paroxetine, reboxetine, sertraline, tianeptine, acetaphenazine, binedaline, brofaromine, cericlamine, clovoxamine, iproniazid, isocarboxazid, moclobemide, phenyhydrazine, phenelzine, selegiline, sibutramine, tranylcypromine, ademetionine, adrafinil, amesergide, amisulpride, amperozide, benactyzine, bupropion, caroxazone, gepirone, idazoxan, metralindole, milnacipran, minaprine, nefazodone, nomifensine, ritanserin, roxindole, S-adenosylmethionine, tofenacin, trazodone, tryptophan, venlafaxine, and zalospirone.
Typically, when the drug is an antiemetic, it is selected from one of the following compounds: alizapride, azasetron, benzquinamide, bromopride, buclizine, chlorpromazine, cinnarizine, clebopride, cyclizine, diphenhydramine, diphenidol, dolasetron methanesulfonate, droperidol, granisetron, hyoscine, lorazepam, metoclopramide, metopimazine, ondansetron, perphenazine, promethazine, prochlorperazine, scopolamine, triethylperazine, trifluoperazine, triflupromazine, trimethobenzamide, tropisetron, domeridone, and palonosetron.
Typically, when the drug is an antihistamine, it is selected from one of the following compounds: azatadine, brompheniramine, chlorpheniramine, clemastine, cyproheptadine, dexmedetomidine, diphenhydramine, doxylamine, hydroxyzine, cetrizine, fexofenadine, loratidine, and promethazine.
Typically, when the drug is an antiparkisonian drug, it is selected one of the following compounds: amantadine, baclofen, biperiden, benztropine, orphenadrine, procyclidine, trihexyphenidyl, levodopa, carbidopa, selegiline, deprenyl, andropinirole, apomorphine, benserazide, bromocriptine, budipine, cabergoline, dihydroergokryptine, eliprodil, eptastigmine, ergoline pramipexole, galanthamine, lazabemide, lisuride, mazindol, memantine, mofegiline, pergolike, pramipexole, propentofylline, rasagiline, remacemide, spheramine, terguride, entacapone, and tolcapone.
Typically, when the drug is an antipsychotic, it is selected from one of the following compounds: acetophenazine, alizapride, amperozide, benperidol, benzquinamide, bromperidol, buramate, butaperazine, carphenazine, carpipramine, chlorpromazine, chlorprothixene, clocapramine, clomacran, clopenthixol, clospirazine, clothiapine, cyamemazine, droperidol, flupenthixol, fluphenazine, fluspirilene, haloperidol, mesoridazine, metofenazate, molindrone, penfluridol, pericyazine, perphenazine, pimozide, pipamerone, piperacetazine, pipotiazine, prochlorperazine, promazine, remoxipride, sertindole, spiperone, sulpiride, thioridazine, thiothixene, trifluperidol, triflupromazine, trifluoperazine, ziprasidone, zotepine, zuclopenthixol, amisulpride, butaclamol, clozapine, melperone, olanzapine, quetiapine, and risperidone.
Typically, when the drug is an anxiolytic, it is selected from one of the following compounds: mecloqualone, medetomidine, metomidate, adinazolam, chlordiazepoxide, clobenzepam, flurazepam, lorazepam, loprazolam, midazolam, alpidem, alseroxlon, amphenidone, azacyclonol, bromisovalum, buspirone, calcium N-carboamoylaspartate, captodiamine, capuride, carbcloral, carbromal, chloral betaine, enciprazine, flesinoxan, ipsapiraone, lesopitron, loxapine, methaqualone, methprylon, propanolol, tandospirone, trazadone, zopiclone, and zolpidem.
Typically, when the drug is a drug for erectile dysfunction, it is selected from one of the following compounds: cialis (IC351), sildenafil, vardenafil, apomorphine, apomorphine diacetate, phentolamine, and yohimbine.
Typically, when the drug is a drug for migraine headache, it is selected from one of the following compounds: almotriptan, alperopride, codeine, dihydroergotamine, ergotamine, eletriptan, frovatriptan, isometheptene, lidocaine, lisuride, metoclopramide, naratriptan, oxycodone, propoxyphene, rizatriptan, sumatriptan, tolfenamic acid, zolmitriptan, amitriptyline, atenolol, clonidine, cyproheptadine, diltiazem, doxepin, fluoxetine, lisinopril, methysergide, metoprolol, nadolol, nortriptyline, paroxetine, pizotifen, pizotyline, propanolol, protriptyline, sertraline, timolol, and verapamil.
Typically, when the drug is a drug for the treatment of alcoholism, it is selected from one of the following compounds: naloxone, naltrexone, and disulfuram.
Typically, when the drug is a drug for the treatment of addiction it is buprenorphine.
Typically, when the drug is a muscle relaxant, it is selected from one of the following compounds: baclofen, cyclobenzaprine, orphenadrine, quinine, and tizanidine.
Typically, when the drug is a nonsteroidal anti-inflammatory, it is selected from one of the following compounds: aceclofenac, alminoprofen, amfenac, aminopropylon, amixetrine, benoxaprofen, bromfenac, bufexamac, carprofen, choline, salicylate, cinchophen, cinmetacin, clopriac, clometacin, diclofenac, etodolac, indoprofen, mazipredone, meclofenamate, piroxicam, pirprofen, and tolfenamate.
Typically, when the drug is an opioid, it is selected from one of the following compounds: alfentanil, allylprodine, alphaprodine, anileridine, benzylmorphine, bezitramide, buprenorphine, butorphanol, carbiphene, cipramadol, clonitazene, codeine, dextromoramide, dextropropoxyphene, diamorphine, dihydrocodeine, diphenoxylate, dipipanone, fentanyl, hydromorphone, L-alpha acetyl methadol, lofentanil, levorphanol, meperidine, methadone, meptazinol, metopon, morphine, nalbuphine, nalorphine, oxycodone, papavereturn, pethidine, pentazocine, phenazocine, remifentanil, sufentanil, and tramadol.
Typically, when the drug is an other analgesic it is selected from one of the following compounds: apazone, benzpiperylon, benzydramine, caffeine, clonixin, ethoheptazine, flupirtine, nefopam, orphenadrine, propacetamol, and propoxyphene.
Typically, when the drug is a stimulant, it is selected from one of the following compounds: amphetamine, brucine, caffeine, dexfenfluramine, dextroamphetamine, ephedrine, fenfluramine, mazindol, methyphenidate, pemoline, phentermine, and sibutramine.

Drugs which would particularly benefit from controlled pulmonary delivery according to the present invention include triptans, citalopram, triamterene, isoniazid, and combinations of various respiratory and systemic drugs. The drug may be a triptan selected from the group consisting of rizatriptan, sumatriptan, naratriptan, zolmitriptan, eletriptan, almotriptan, and frovatriptan.

For purposes of modulating dissolution and/or pharmacokinetic absorption of the drug, the second compoundis typically selected from the group consisting of long-chain fatty acids, alcohols, amines, and hydrocarbons; for example and not by way of limitation, palmitic acid, hexadecanol, hexadecyl amine, and/or hexadecane may be used.

For purposes of altering vaporization characteristics, high vapor pressure or sublimable second compounds, such as maltol, benzoic acid, caffeine, fumaric acid, norvaline, and/or menthol, for example and not by way of limitation, may be used.

For purposes of improving drug palatability of undesirable tasting drugs, taste attenuating agents such as sweeteners (e.g., acesulfame, xylitol), menthol, and/or flavoring agents (e.g., strawberry furanone), for example and not by way of limitation, may be used.

### EXPERIMENTAL EXAMPLES

The following experimental examples further illustrate the method and various embodiments of the present invention. These examples are for illustrative purposes and are not meant to limit the scope of the claims in any way.

### Example One

### Generation of Heterogeneous Particles of Rizatriptan and Palmitic Acid

An aerosol comprising heterogeneous particles of rizatriptan (free-based from rizatriptan benzoate salt, obtained from Topharman, Shanghai, China) and palmitic acid (obtained from CalBioChem/EMD Biosciences, San Diego, Calif.) was generated as follows: A heating substrate (foil) was coated with a solution comprising riztariptan free-base and palmitic acid in organic solvent. After the solvent evaporated, a thin film remained on the substrate. Upon rapid heating of the heating substrate in the presence of airflow across the substrate, the thin film vaporized and condensed to form an aerosol. Brownian motion, flow discontinuities, and chemical attractions all facilitate the mixing of the first (drug) compound and the second (excipient) compound in the vapor phase so that upon condensation, heterogeneous aerosol particles may be formed.

Palmitic acid (chemical formula: C₁₆H₃₂O₂; chemical name: hexadecanoic acid; other names: hexadecyclic acid, cetylic acid) is one of the most common saturated fatty acids found in animals and plants. Palmitic acid is a major compound of the oil from palm trees (palm oil and palm kernel oil). Palmitic acid (which is also found in butter, cheese, milk, and meat) was selected as the second compound (excipient) in this experiment due to its relative physiological abundance.

### Example Two

### Scanning Electron Microscopy of Heterogeneous Particles of Rizatriptan and Palmitic Acid

Scanning electron microscopy (SEM) of heterogeneous particles of rizatriptan and palmitic acid (generated as described in Example One, above) was performed using a Philips XL-30FEG scanning electron microscope (Philips Electronics, Amsterdam) at a magnification level of 550×.

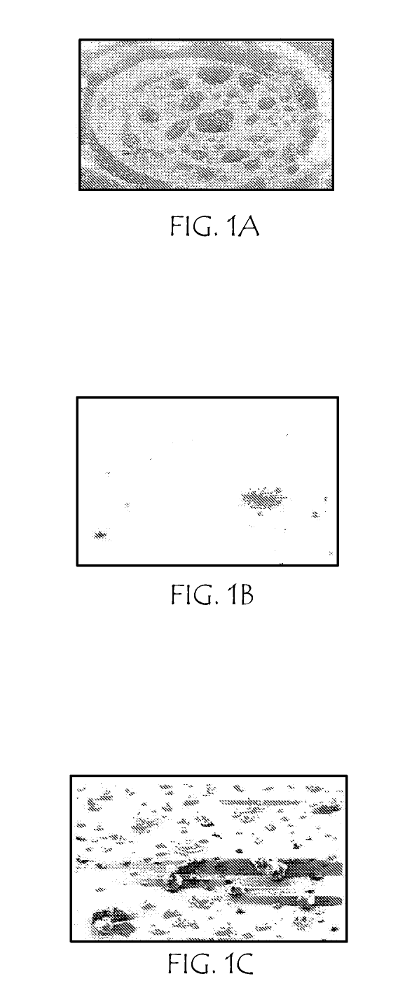

FIG. 1A is an SEM photomicrograph of unary (single-compound) particles of rizatriptan aerosol; FIG. 1B is an SEM photomicrograph of unary particles of palmitic acid aerosol; FIG. 1C is an SEM photomicrograph of heterogeneous particles of rizatriptan and palmitic acid (3:1 mole ratio of palmitic acid:rizatriptan) aerosol.

From the morphology of the respective unary aerosol particles, SEM imaging showed good mixing of drug and excipient.

### Example Three

### Raman Spectroscopy of Heterogeneous Particles of Rizatriptan and Palmitic Acid

Raman spectroscopy is the collection of light inelastically scattered by a material or compound. When a light of known wavelength strikes a material, the light is shifted according to the chemical functionalities of the material. The intensity of this shifted light depends on both the molecular structure and macrostructure of the material. As a result of these phenomena, the collection of the shifted light gives a Raman spectrum that can provide direct information regarding the molecular vibrations of the compound or material. This information can then be interpreted to determine chemical structure, organization and, in some cases, non-covalent intermolecular interactions.

In order to confirm that individual particles generated by the present method contain both drug and excipient, Raman spectroscopy analysis of heterogeneous particles of rizatriptan and palmitic acid was performed by Evans Analytical Group (Sunnyvale, Calif.). The measurements were performed using a “LabRam” J-Y Spectrometer equipped with a 600 gr/mm grating. A HeNe laser (632.817 nm wavelength) was used as the excitation source. The measurements were performed under an Olympus BX40 microscope (Olympus America, Center Valley, Pa.).

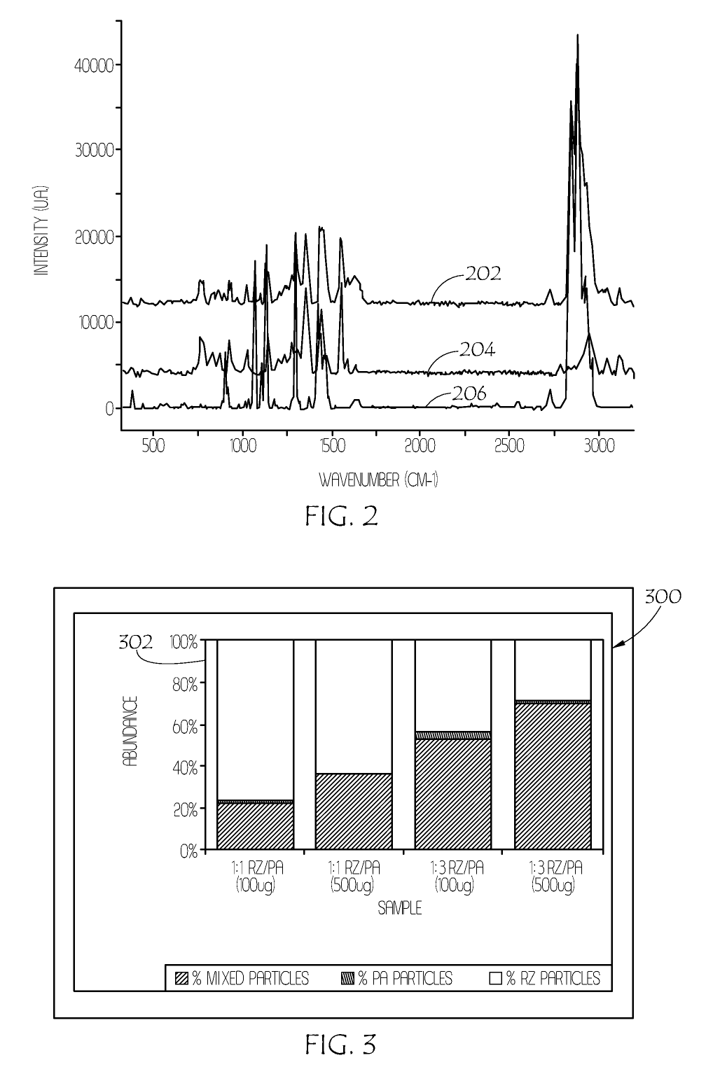

The particles were gravitationally settled onto glass slides. Unary (single-compound) particles of rizatriptan, unary particles of palmitic acid, and heterogeneous particles of palmitic acid and rizatriptan (from vaporization of a 3:1 mole ratio of palmitic acid:rizatriptan) were probed. FIG. 2 compares the reference spectra of single-compound particles of rizatriptan 204 and palmitic acid 206 with the spectrum 202 obtained from heterogeneous particles of palmitic acid and rizatriptan, and clearly demonstrates that such particles contain a mixture of drug and excipient. The rizatriptan spectrum 204 has a peak at 1545 cm⁻¹ due to ring vibration of the drug, which does not overlap with the bands of palmitic acid and can be used to identify the drug presence in the particles of mixture. The palmitic acid spectrum 206 has the stretching vibration of a long CH₂ chain at 2840 cm⁻¹ (symmetric) and 2878 cm⁻¹ (antisymmetric), which can be used to identify the presence of the palmitic acid.

### Example Four

### Particle Size Analysis of Heterogeneous Particles of Rizatriptan and Palmitic Acid

Aerodynamic particle sizing and laser desorption/ionization (LDI) of heterogeneous particles of palmitic acid and rizatriptan was conducted by TSI Incorporated (Shoreview, Minn.) using Aerosol Time-of-Flight Mass Spectrometry (ATOFMS). ATOFMS utilizes an aerodynamic time-of-flight sizing technique to size individual particles in near real time. Single particle laser desorption/ionization facilitates chemical analysis in a bipolar, time-of-flight mass spectrometer. (See U.S. Pat. Nos. 5,681,752 and 5,998,215.)

Heterogeneous aerosol particles of palmitic acid and rizatriptan, at palmitic acid rizatriptan mole ratios of 1:1 and 3:1, and drug loading of 100 μg and 500 μg rizatriptan, were analyzed. Vaporization temperature was 350° C. for all samples tested.

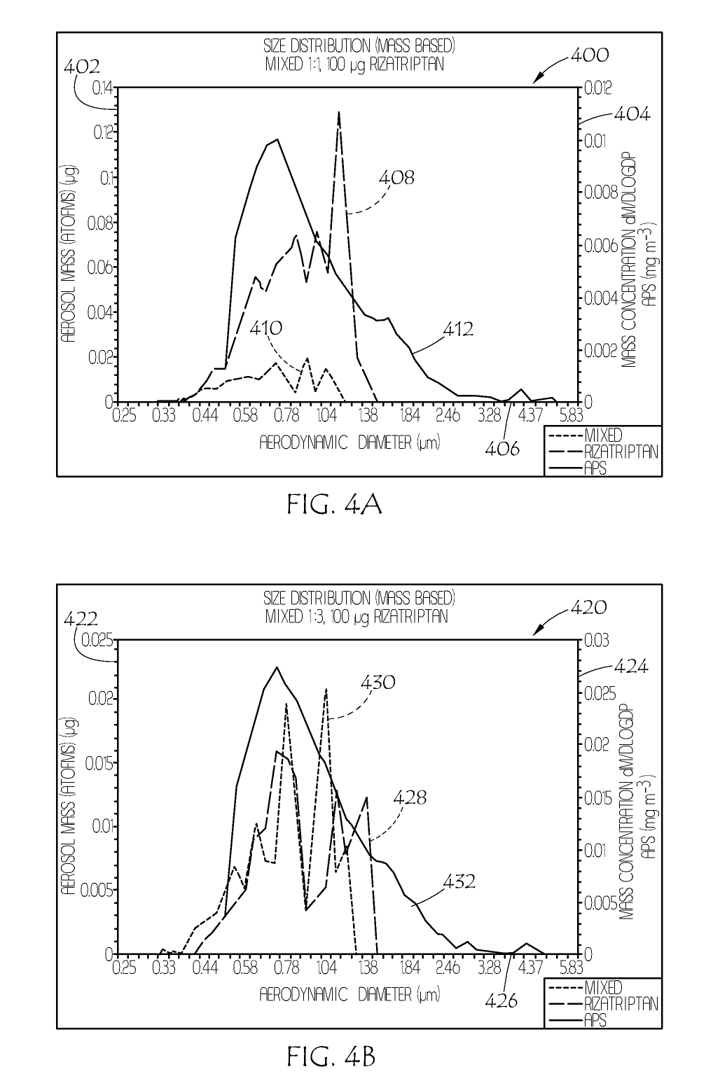

FIG. 3 is a bar graph 300 showing the laser desorption/ionization (ATOFMS-LDI) fractions 302 of heterogeneous particles in samples generated from a 1:1 mole ratio of palmitic acid:rizatriptan, 100 and 500 μg drug loading; and a 3:1 mole ratio of palmitic acid:rizatriptan, 100 and 500 μg drug loading.

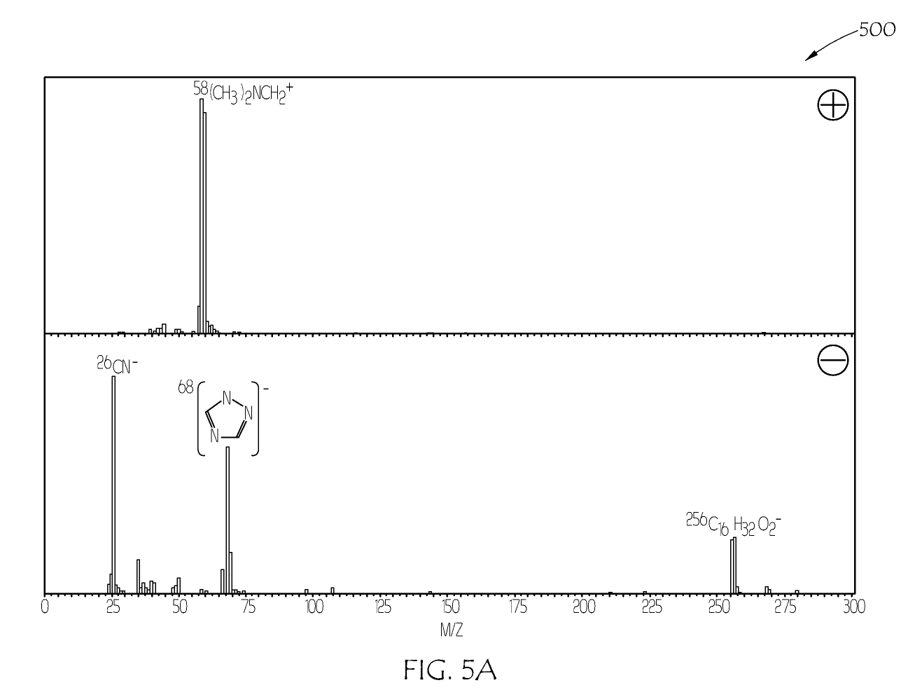

FIG. 4A is a line graph 400 showing the mass-based size distributions of the aerosol particles acquired from ATOFMS 402 for rizatriptan 408 and heterogeneous particles of palmitic acid:rizatriptan 410 (1:1 mole ratio of palmitic acid:rizatriptan, 100 μg drug loading), and APS (total size distribution) 404 for the heterogeneous particles 412, as a function of aerodynamic diameter 406.

FIG. 4B is a line graph 420 showing the mass-based size distributions of the aerosol particles acquired from ATOFMS 422 for rizatriptan 428 and heterogeneous particles of palmitic acid:rizatriptan 430 (3:1 mole ratio of palmitic acid:rizatriptan, 100 μg drug loading), and APS (total size distribution) 424 for the heterogeneous particles 432, as a function of aerodynamic diameter 426.

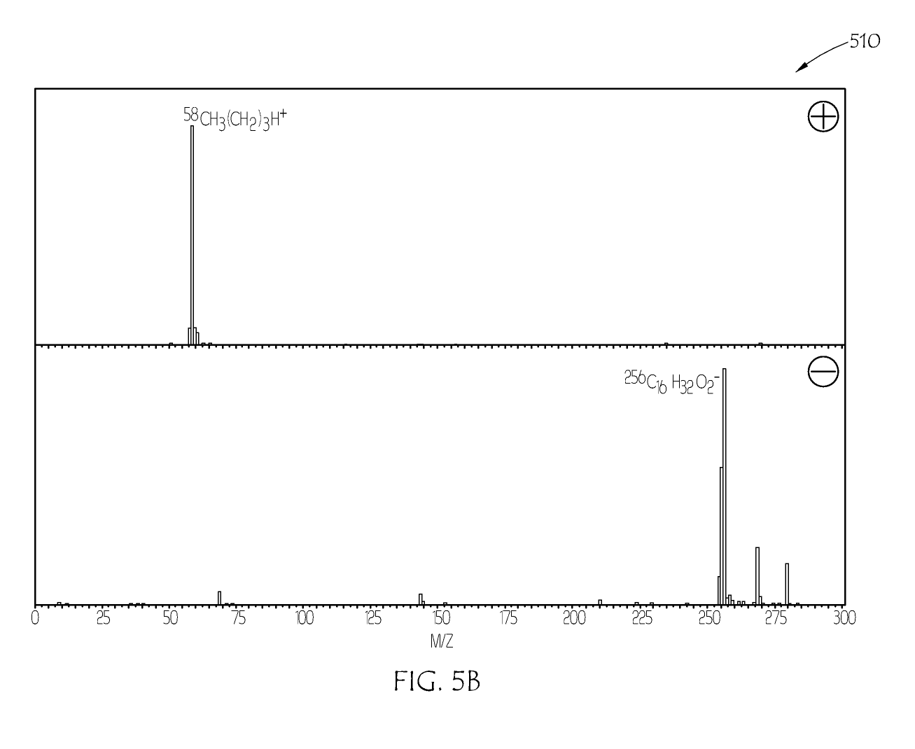

FIG. 5A is an ATOFMS spectrum 500 generated from a rizatriptan aerosol sample. FIG. 5B is an ATOFMS spectrum 510 generated from a palmitic acid aerosol sample. FIG. 5C is an ATOFMS spectrum 520 generated from a 3:1 palmitic acid rizatriptan aerosol sample.

The LDL data showed that at the higher 3:1 mole ratio of palmitic acid to rizatriptan, the fraction (yield) of heterogeneous particles increased in comparison to the 1:1 mole ratio (approximately 70% compared to 35%, respectively, for the 500 μg drug loading). The mass distribution of the particle sizes is fairly consistent with modes focusing at approximately 1 μm.

### Example Five

### Scanning Electron Microscopy of Heterogeneous Particles of Prochlorperazine and Acesulfame

Acesulfame (chemical formula: C₄H₅NO₄S) is a common synthetic, normutritive sweetener used in foods and cosmetics. It was selected as an appropriate excipient to attenuate the taste of PCZ based on its GRAS (generally recognized as safe) status and evidence that suggests it reduces the throat irritation associated with nicotine (see, for example, U.S. Patent Publication No. 2004/0173224).

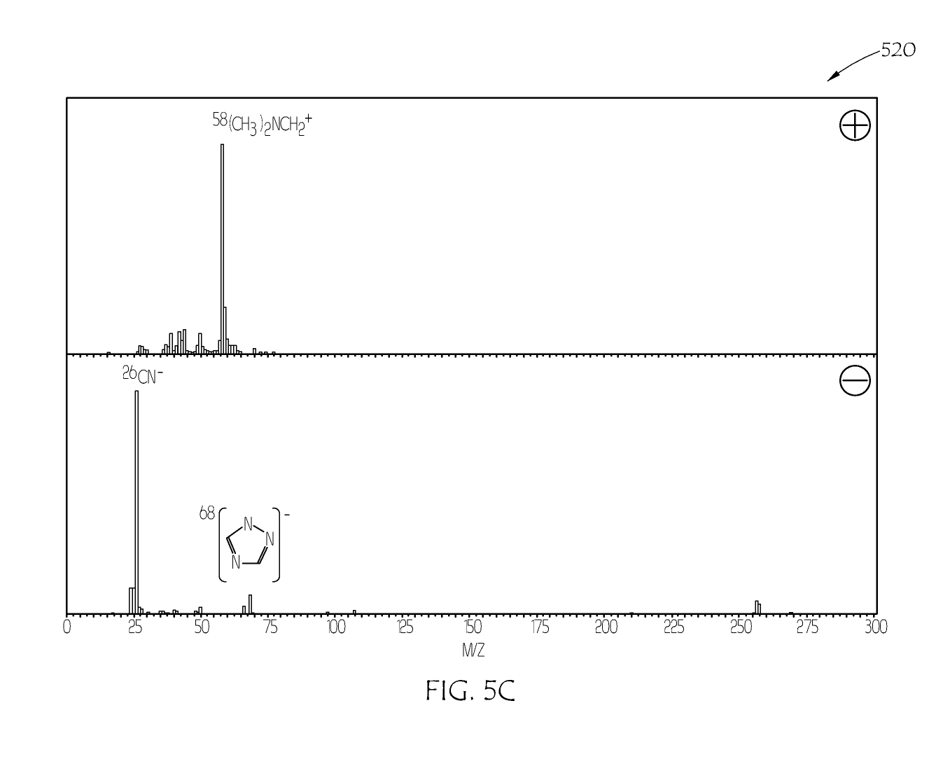

FIGS. 6A-6C show three different coating configurations that can be used to co-vaporize a first compound (drug) and a second compound from a heating substrate.

FIG. 6A shows a drug and a second compound coated on the same surface of a heating substrate, with the drug at the trailing edge of airflow across the substrate. FIG. 6B shows a drug and a second compound coated on the same surface of a heating substrate, with the drug at the leading edge of airflow across the substrate. FIG. 6C shows a drug coated onto the top surface of the substrate and a second compound coated onto the bottom surface of the substrate.

An aerosol composition comprising prochlorperazine (PCZ) and acesulfame (ACE, free acid obtained from the potassium salt of acesulfame) was generated as follows: A portion of a heating substrate (foil) was coated with a solution of PCZ dissolved in acetone. After the solvent evaporated, a different portion of the same substrate was coated with a solution of ACE dissolved in 3:1 dichloromethane:acetone. After all of the solvent evaporated, a thin film remained on the substrate. Upon rapid heating of the heating substrate in the presence of airflow across the substrate, the thin film vaporized and condensed to form an aerosol. Brownian motion, flow discontinuities, and chemical attractions all facilitate the mixing of the drug compound and the excipient in the vapor phase so that upon condensation, heterogeneous aerosol particles may be formed.

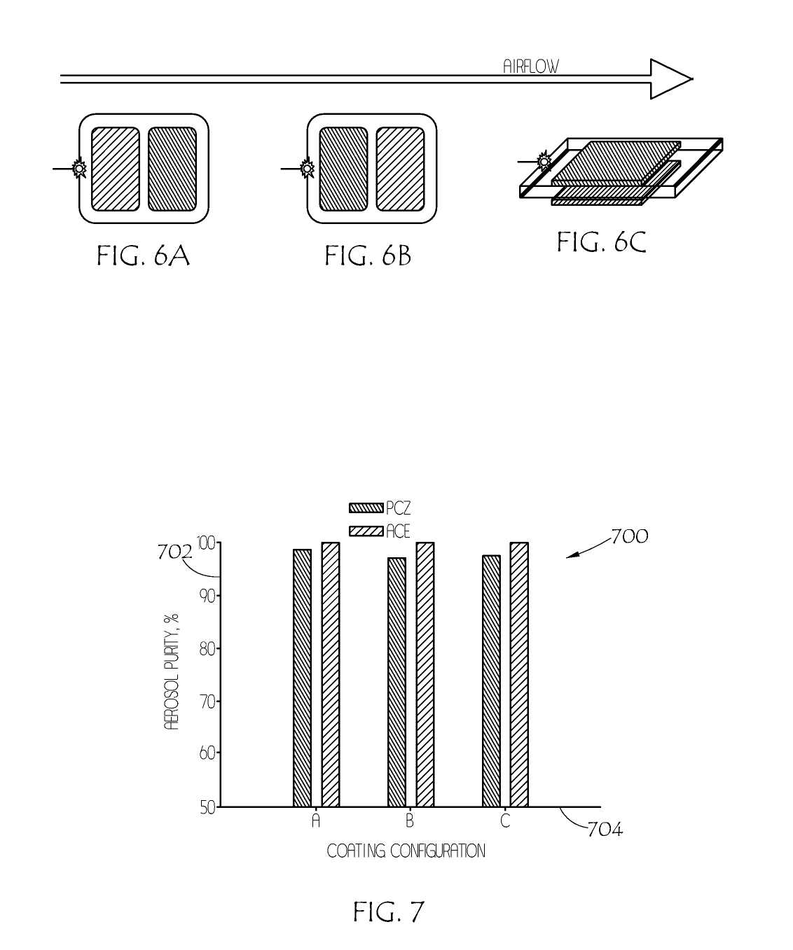

FIG. 7 is a bar graph 700 showing aerosol purity 702 of prochlorperazine (PCZ) and acesulfame (ACE) when vaporized from a heating substrate in three different coating configurations 704, as follows:

A: PCZ coated on the top surface of the substrate and ACE coated on the bottom surface of the substrate (illustrated in FIG. 6C);

B: PCZ and ACE coated on the same surface of the substrate, with PCZ at the leading edge of airflow across the substrate (illustrated in FIG. 6B);

C: PCZ and ACE coated on the same surface of the substrate, with PCZ at the trailing edge of airflow across the substrate (illustrated in FIG. 6C).

FIG. 7 illustrates that PCZ can be co-vaporized in the presence of ACE without significantly affecting the purity of the aerosolized PCZ.

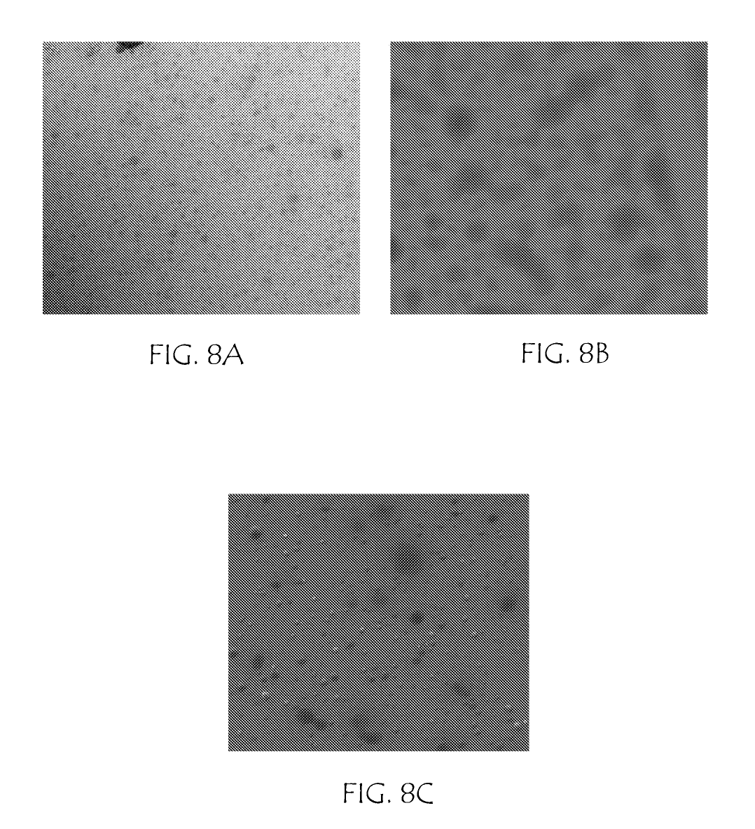

Scanning electron microscopy (SEM) of single-compound particles of ACE and PCZ, and heterogeneous particles of PCZ and ACE (generated as described above) was performed using a Philips XL-30FEG scanning electron microscope (Philips Electronics, Amsterdam) at a magnification level of 1000×. FIG. 8A is an SEM photomicrograph of unary (single-compound) particles of ACE. FIG. 8B is an SEM photomicrograph of unary (single-compound) particles of PCZ. FIG. 8C is an SEM photomicrograph of heterogeneous particles of PCZ and ACE (1:1 mole ratio) vaporized from a heating substrate with the coating configuration shown in FIG. 6C.

The morphologies of the PCZ particles and the ACE particles are shown in FIGS. 8A and 8B. FIG. 8C showed good mixing of PCZ and ACE.

Heterogeneous particles potentially allow the modification of a drug's pharmacokinetic profile and taste. The characterization techniques described in the above Examples facilitate the study of new formulation approaches directed toward taste attenuation and improving the vaporizability of aerosolized drug formulations, as well as the development of combination drug therapies.

One of ordinary skill in the art can combine the foregoing embodiments or make various other embodiments and aspects of the method and device of the present invention to adapt them to specific usages and conditions. As such, these changes and modifications are properly, equitably, and intended to be within the full range of equivalents of the following claims.

We claim:

1. A aerosol comprising a first compound which is physiologically active, and a second compound which is different from the first compound, wherein said aerosol comprises aerosolized particles, wherein at least 10% of said aerosolized particles comprise both said first compound and said second compound said aerosol, and wherein said aerosol has amass median aerodynamic diameter (MMAD) in the range of 0.1 μm to 20 μm.

2. The drug delivery composition of claim 1, wherein at least 25% of said aerosolized particles comprise both said first compound and said second compound.

3. The drug delivery composition of claim 2, wherein at least 50% of said aerosolized particles comprise both said first compound and said second compound.

4. The drug delivery composition of claim 3, wherein at least 90% of said aerosolized particles comprise both said first compound and said second compound.

5. The drug delivery composition of claim 1, wherein said aerosol has an MMAD within the range of 0.5 μm to 10 μm.

6. The drug delivery composition of claim 5, wherein said aerosol has an MMAD within the range of 1 μm to 5 μm.

7. The drug delivery composition of claim 1, wherein said first compound is selected from the group consisting of a triptan, citalopram, triamterene, isoniazid, and combinations thereof.

8. The drug delivery composition of claim 7, wherein said first compound is a triptan.

9. The drug delivery composition of claim 8, wherein said triptan is selected from the group consisting of rizatriptan, sumatriptan, naratriptan, zolmitriptan, eletriptan, almotriptan, and frovatriptan.

10. The drug delivery composition of claim 1, wherein said second compound is a physiologically inert compound.

11. The drug delivery composition of claim 10, wherein said physiologically inert compound modulates the pharmacokinetic absorption of said first, physiologically active compound.

12. The drug delivery composition of claim 11, wherein said physiologically inert compound is selected from the group consisting of long-chain fatty acids, alcohols, amines, hydrocarbons, and combinations thereof.

13. The drug delivery composition of claim 12, wherein said physiologically inert compound is selected from the group consisting of palmitic acid, hexadecanol, hexadecyl amine, hexadecane, and combinations thereof.

14. The drug delivery composition of claim 10, wherein said physiologically inert compound improves the vaporizability of said first compound.

15. The drug delivery composition of claim 10, wherein said physiologically inert compound is selected from the group consisting of maltol, benzoic acid, caffeine, fumaric acid, norvaline, and menthol.

16. The drug delivery composition of claim 10, wherein said physiologically inert compound is a taste attenuating agent.

17. The drug delivery composition of claim 10, wherein said taste attenuating agent is selected from the group consisting of a sweetener, a flavoring agent, and menthol.

18. The drug delivery composition of claim 1, wherein said second compound is a physiologically active compound which is different from said first compound.

19. A method of producing a heterogeneous aerosolized drug delivery composition containing a first compound which is physiologically active and a second compound which is different from said first compound, wherein said method comprises the steps of:
a) providing a heating substrate;
b) coating at least a portion of a surface of said heating substrate with said first compound and said second compound; and
c) heating said substrate surface to a temperature sufficient to vaporize said first compound and said second compound, whereby an aerosolized drug delivery composition comprising particles is produced.

20. The method of claim 19, wherein said first compound and said second compound are simultaneously coated onto the same or separate areas said heating substrate surface.

21. The method of claim 19, wherein said first compound and said second compound are sequentially coated onto the same or separate areas of said heating substrate surface.

22. The method of claim 19, wherein said first compound and said second compound are coated onto said heating substrate surface using a coating method selected from the group consisting of spray coating, dipcoating, and inkjet printing.

23. The method of claim 22, wherein said first compound and said second compound are coated onto said heating substrate surface by ultrasonic spray coating.

24. The method of claim 19, wherein said substrate surface is heated to a temperature of at least 200° C.

25. The method of claim 24, wherein said substrate surface is heated to a temperature of at least 300° C.

26. The method of claim 25, wherein said substrate surface is heated to a temperature within the range of about 300° C. to about 450° C.

27. The method of claim 19, wherein said substrate surface is heated by electrical, chemical, or electrochemical heating means.

28. The method of claim 19, wherein said first compound is selected from the group consisting of a triptan, citalopram, triamterene, isoniazid, and combinations thereof.

29. The method of claim 28, wherein said first compound is a triptan.

30. The method of claim 29, wherein said triptan is selected from the group consisting of rizatriptan, sumatriptan, naratriptan, zolmitriptan, eletriptan, almotriptan, and frovatriptan.

31. The method of claim 19, wherein said second compound is a physiologically inert compound.

32. The method of claim 31, wherein said second compound and said first compound are coated onto said heating substrate surface at a mole ratio within the range of 1:10 to 10:1 (second compound:first compound).

33. The method of claim 32, wherein said second compound and said first compound are coated onto said heating substrate surface at a mole ratio within the range of about 1:5 to about 5:1 (second compound:first compound).

34. The method of claim 33, wherein said second compound and said first compound are coated onto said heating substrate surface at a mole ratio within the range of about 1:1 to about 3:1 (second compound:first compound).

35. The method of claim 31, wherein said physiologically inert compound modulates the pharmacokinetic absorption of said first, physiologically active compound.

36. The method of claim 35, wherein said physiologically inert compound is selected from the group consisting of long-chain fatty acids, alcohols, amines, hydrocarbons, and combinations thereof.

37. The method of claim 36, wherein said physiologically inert compound is selected from the group consisting of palmitic acid, hexadecanol, hexadecyl amine, hexadecane, and combinations thereof.

38. The method of claim 31, wherein said physiologically inert compound improves the vaporizability of said first, physiologically active compound.

39. The method of claim 38, wherein said physiologically inert compound is selected from the group consisting of maltol, benzoic acid, caffeine, fumaric acid, norvaline, and menthol.

40. The method of claim 30, wherein said physiologically inert compound is a taste attenuating agent.

41. The method of claim 39, wherein said taste attenuating agent is selected from the group consisting of a sweetener, a flavoring agent, and menthol.

42. The method of claim 18, wherein said second compound is a physiologically active compound which is different from said first physiologically active compound.

43. The method of claim 19, wherein at least 10% of said aerosolized particles comprise both said first compound and said second compound.

44. The method of claim 43, wherein at least 25% of said aerosolized particles comprise both said first compound and said second compound.

45. The method of claim 44, wherein at least 50% of said aerosolized particles comprise both said first compound and said second compound.

46. The method of claim 45, wherein at least 90% of said aerosolized particles comprise both said first compound and said second compound.
